(19) United States
(12) Patent Application Publication
(10) Pub. No.: US 2005/0238672 A1
(43) Pub. Date: Oct. 27, 2005

(54) Antifungal drug delivery

(76) Inventor: Marcel E. Nimni, Santa Monica, CA (US)

Correspondence Address: CATALYST LAW GROUP, APC, 9710 SCRANTON ROAD, SUITE S-170, SAN DIEGO, CA, 92121, US

(21) Appl. No.: 10/832,933

(22) Filed: Apr. 27, 2004

(51) Int. Cl. A61K 31/137; A61K 9/00

(52) U.S. Cl. 424/400; 514/649

(57) ABSTRACT

An improved antifungal composition for topical application to the skin and nails comprises: (1) an allylamine antifungal compound; (2) an aliphatic alcohol substituted with an aromatic substituent in which the allylamine antifungal compound is soluble to a degree that a therapeutically effective concentration of the allylamine antifungal compound can be applied topically in solution; (3) a lower aliphatic alcohol in which the aromatic alcohol is soluble; and (d) water or a water-compatible solvent mixture. The allylamine antifungal compound can be terbinafine or naftifine. The aliphatic alcohol substituted with an aromatic substituent can be benzyl alcohol or phenethyl alcohol. The lower aliphatic alcohol can be ethyl alcohol or isopropyl alcohol. In an alternative, the composition can further comprise an additional antifungal compound. Another aspect of the invention is a method for treatment of a fungal infection of skin or nails comprising administering the antifungal composition of the present invention topically to the skin or nails in an amount therapeutically effective to treat the fungal infection.

### BACKGROUND OF THE INVENTION

The present invention generally relates to a method of antifungal drug delivery, especially to the delivery of hydrophobic antifungal compounds such as terbinafine.

The fingernails and toenails are susceptible to dematophytic infections caused by the invasion of fungi into the nails of human beings and other animals. There are numerous fungi, such as Trichophyton rubrum, Microsporum canis, T. mentagrophytes, T. interdigitale, and other known fungi that can cause such infections. Their treatment, particularly when it involves the nails, requires the oral administration of one or more known antifungal agents, e.g. griseofulvin, ketoconazole, terbinafine, ciclopirox olamine, and other agents. The general rule is that if these infections are not treated early they become difficult to combat, and that even if oral administration results in clearing the disease, recurrence is common. In addition, many of these compounds, because they are poorly absorbed from the gastrointestinal tract, have to be administered in relatively large amounts, and for prolonged periods of time, up to one year, to saturate the infected site and be effective.

Despite the fungicidal effectiveness of many of the newer compounds, there is always a concern regarding toxicity, carcinogenicity, and side effects, which require the patients be monitored periodically when treated orally. In particular, many of these compounds can affect liver function. Basic liver function studies and white cell counts are usually routinely performed, adding considerably to the costs of treatment and alarming patients. It is generally believed that risks can be reduced considerably if these compounds could be administered topically at the site of the infection, rather than allowing it to reach such sites via the systemic circulation.

The topical administration of these water insoluble compounds, particularly terbinafine, has been hindered by the lack of a suitable carrier. The keratinized nails, in contrast to the keratinized stratum corneum of the skin, are readily permeable to water and resist the diffusion of hydrophobic compounds. Terbinafine, a highly water insoluble compound, which in addition is insoluble in most organic solvents, is incapable of permeating through the nail plate. This explains why nail lacquers, solutions in organic solvents, and suspensions that contain this compound are ineffective topically in the treatment of onychomycosis.

In applications such as these it is obviously desirable to be able to topically apply pharmaceutically active compounds directly to affected areas. If the medication in question does not penetrate the upper surfaces of the skin or nails, which consists of dead keratinocytes and the fibrous protein keratin, and just deposits on the surface, it can be readily removed by friction, -washing or by the normal detachment of the keratinized epithelium. However, as indicated above, it has proven very difficult to apply such antifungal compounds directly to the affected areas for optimum therapeutic response.

Various compositions for application to or treatment of nails are known, including nail polishes, nail polish removers, nail oil emulsions, nail penetration enhancers, nail softeners, and the like have been developed. However, these have the problems described above.

U.S. Pat. No. 3,382,151 describes an aqueous-based, formaldehyde containing composition which can be applied to finger nails to strengthen them. The patent further discloses that the product has aseptic properties, curing fungi which occasionally infect nails, but which also causes some inflammation.

U.S. Pat. No. 4,820,724 describes a dual phase solvent carrier system for topically applying at least one pharmaceutically active compound comprised of the active compound dissolved in at least one delivery solvent and at least one fugitive solvent, with a particularly useful composition for topically treating dermatophytic infections comprised of griseofulvin, benzyl alcohol and at least one fugitive solvent.

U.S. Pat. No. 4,957,730 describes a nail varnish comprising a water insoluble film forming substance and a series of antimycotic compounds derived from a 1-hydroxy-2-pyridone structure.

U.S. Pat No. 6,495,124 describes a lacquer for treating or preventing fungal infections which includes several known antifungal agents entrapped in a water-insoluble film forming polymer, pentadecalactone, which also is claimed to act as a penetration enhancer.

U.S. Pat. No. 6,380,236 describes the use of a tissue softening composition containing urea and an antifungal composition concurrently or non-concurrently. The kit also includes the use of a protective gel-dressing for ready application.

U.S. Pat. No. 6,042,845 discloses a method for treating fungal diseases of the nail which includes the use of sulfhydryl containing amino acids and urea as permeation enhancers of antifungal drugs.

U.S. Pat. No. 5,889,039 describes the use of a topical antifungal preparation which contains either sulconazole or naftifine combined with an acetate penetration enhancer.

Many of the above inventions are less effective because the lack of water solubility of the very hydrophobic antifungal agents.

This limitation may also apply to the technology described in U.S. Pat. No. 5,487,776, which although disclosing a method for solubilizing griseofulvin, did not allow for the solution to be compatible with water. We believe that the more limited benefits afforded by our previous formulation was probably due in great part to the fact than when the lacquer containing griseofulvin was applied to the surface of the nail, the presence of water in the nail plate was sufficient to cause the precipitation of the antifungal and thus impede its penetration across the nail plate to the underlying nail bed.

Therefore while certain antifungal agents may be applied topically or orally, most have only found to be effective orally in treating infections of the highly keratinized areas of the skin. When nails are infected, particularly toenails, oral administration has proven to be the only effective way of treatment. A major incentive for topical application is that total amounts of drug used for this route is several orders of magnitude less, and the effects are local. This offers a very significant margin of safety over oral administration. Unfortunately topical administration of these agents, with concomitant effectiveness, has been hindered by the lack of a suitable carrier or modality of penetration route that enhances the actual solubility of the otherwise water insoluble drugs.

Therefore, there is a need for an improved carrier and method for antifungal drug delivery so that such drugs can be topically delivered, especially to the nails. The improved carrier and method for antifungal drug delivery are particularly desirable for the delivery of drugs such as terbinafine.

### SUMMARY OF THE INVENTION

In general, an antifungal composition according to the invention for topical application to the skin and nails comprises:
(1) an allylamine antifungal compound;
(2) an aliphatic alcohol substituted with an aromatic substituent in which the allylamine antifungal compound is soluble to a degree that a therapeutically effective concentration of the allylamine antifungal compound can be applied topically in solution;
(3) a lower aliphatic alcohol in which the aromatic alcohol is soluble; and
(4) water or a water-compatible solvent mixture.

Typically, the allylamine antifungal compound is terbinafine. However, the allylamine antifungal compound can be another allylamine such as naftifine or an analogue or derivative of terbinafine or naftifine.

Typically, the aliphatic alcohol substituted with an aromatic substituent is benzyl alcohol. However, in another alternative, the aromatic alcohol is phenethyl alcohol or another aromatic alcohol.

Typically, the lower aliphatic alcohol is selected from the group consisting of ethyl alcohol, isopropyl alcohol, and mixtures thereof. Preferably, the lower aliphatic alcohol is ethyl alcohol. More preferably, the ethyl alcohol is absolute ethyl alcohol. Alternatively, the ethyl alcohol can be 95% ethyl alcohol. In another alternative, the lower aliphatic alcohol can be a mixture of ethyl alcohol and isopropyl alcohol.

Typically, the concentration of the allylamine antifungal compound is from about 1% (w/v) to about 3% (w/v) in the final composition. Preferably, the concentration of the allylamine antifungal compound is from about 1.5% (w/v) to about 2.5% (w/v) in the final composition. More preferably, the concentration of the allylamine antifungal compound is about 2% in the final composition.

Typically, the concentration of the aliphatic alcohol substituted with an aromatic substituent is from about 3% (v/v) to about 10% (v/v) in the final composition. Preferably, the concentration of the aliphatic alcohol substituted with an aromatic substituent is about 5% (v/v) in the final composition.

Typically, the concentration of the lower aliphatic alcohol is from about 80% (v/v) to about 95% (v/v). Preferably, the concentration of the lower aliphatic alcohol is from about 82.5% (v/v) to about 87.5% (v/v). More preferably, the concentration of the lower aliphatic alcohol is about 85% (v/v).

Typically, the concentration of water or the water-compatible solvent mixture is from about 1% (v/v) to about 15% (v/v). Preferably, the concentration of water or the water-compatible solvent mixture is from about 9% (v/v) to about 11% (v/v). More preferably, the concentration of water or the water-compatible solvent mixture is about 10% (v/v). Typically, water is used; alternatively, a water-compatible solvent mixture can be used. This would comprise hydrophilic polar organic solvents.

In another embodiment, the invention comprises an additional antifungal compound. In general, this embodiment comprises:
(1) an allylamine antifungal compound;
(2) an additional antifungal compound;
(3) an aliphatic alcohol substituted with an aromatic substituent in which the allylamine antifungal compound and the at least one additional antifungal compound are soluble to a degree that a therapeutically effective concentration of the allylamine antifungal compound and of the additional antifungal compound can be applied topically in solution;
(4) a lower aliphatic alcohol in which the aromatic alcohol is soluble; and
(5) water or a water-compatible solvent mixture.

The additional antifungal compound can be any of griseofulvin, ketoconazole, griseofulvin, miconazole, itraconazole, fluconazole, clotrimazole, econazole, terconazole, butoconazole, tioconazole, oxiconazole, sulconazole, cicloprox olamine, haloprogin, and tolnaftate. Typically, the additional antifungal compound is griseofulvin, miconazole, ketoconazole, both griseofulvin and ketoconazole, or both griseofulvin and miconazole. The compound or mixture of compounds should remain soluble in the presence of 10% to 20% water.

In this embodiment, the concentration of the allylamine antifungal compound is preferably about 2% (w/v). The concentration of the additional antifungal compound is typically from about 1% (w/v) to about 3% (w/v), preferably from about 1.5% (w/v) to about 2.5% (w/v), more preferably about 2% (w/v). Other concentrations can be used depending on the specific additional antifungal compounds included. Typically, the volume of the lower aliphatic alcohol used is adjusted to take the concentration of the additional antifungal compound into account.

Another embodiment of the invention is a method of treating a fungal infection of skin or nails, particularly onychomycosis. The method comprises topically administering a composition according to the present invention to the skin or nails in an amount therapeutically effective to treat the fungal infection. In particular, the fungal infection is an infection of the plantar or peri-plantar regions of the foot or of the subungual epithelium present above and around the nail bed. The fungal infection can be an infection caused by Trichophyton rubrum, Microsporum canis, T. mentagrophytes, T. interdigitale, or another fungal species.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

One embodiment of the present invention is an antifungal composition for topical application to the skin and nails. In general, this embodiment of the composition comprises:
(1) an allylamine antifungal compound;
(2) an aliphatic alcohol substituted with an aromatic substituent in which the allylamine antifungal compound is soluble to a degree that a therapeutically effective concentration of the allylamine antifungal compound can be applied topically in solution;
(3) a lower aliphatic alcohol in which the aromatic alcohol is soluble; and
(4) water or a water-compatible solvent mixture.

Typically, the allylamine antifungal compound is terbinafine. However, the allylamine antifungal compound can be another allylamine such as naftifine or an analogue or derivative of terbinafine or naftifine.

Typically, the aliphatic alcohol substituted with an aromatic substituent is benzyl alcohol. However, in another alternative, the aromatic alcohol is phenethyl alcohol or another aromatic alcohol.

Typically, the lower aliphatic alcohol is selected from the group consisting of ethyl alcohol, isopropyl alcohol, and mixtures thereof. Preferably, the lower aliphatic alcohol is ethyl alcohol. More preferably, the ethyl alcohol is absolute ethyl alcohol. Alternatively, the ethyl alcohol can be 95% ethyl alcohol. In another alternative, the lower aliphatic alcohol can be a mixture of ethyl alcohol and isopropyl alcohol.

Typically, the concentration of the allylamine antifungal compound is from about 1% (w/v) to about 3% (w/v) in the final composition. Preferably, the concentration of the allylamine antifungal compound is from about 1.5% (w/v) to about 2.5% (w/v) in the final composition. More preferably, the concentration of the allylamine antifungal compound is about 2% in the final composition.

Typically, the concentration of the aliphatic alcohol substituted with an aromatic substituent is from about 3% (v/v) to about 10% (v/v) in the final composition. Preferably, the concentration of the aliphatic alcohol substituted with an aromatic substituent is about 5% (v/v) in the final composition.

Typically, the concentration of the lower aliphatic alcohol is from about 80% (v/v) to about 90% (v/v). Preferably, the concentration of the lower aliphatic alcohol is from about 82.5% (v/v) to about 87.5% (v/v). More preferably, the concentration of the lower aliphatic alcohol is about 85% (v/v).

Typically, the concentration of water or the water-compatible solvent mixture is from about 1% (v/v) to about 12.5% (v/v). Preferably, the concentration of water or the water-compatible solvent mixture is from about 9% (v/v) to about 11% (v/v). More preferably, the concentration of water or the water-compatible solvent mixture is about 10% (v/v). Typically, water is used; alternatively, a water-compatible solvent mixture can be used. This would comprise hydrophilic polar organic solvents.

The invention is directed at the delivery of drugs that have to act on the surface of the skin or under the nail, and that have to be retained for prolonged periods of time stored in the interstices of the epidermis, at the epidermal-dermal junction or sub-epidermal regions. In particular its focus is on the delivery of antifungal agents that are required to act in the interstices of highly keratinized epithelium, such as that encountered on the plantar and peri-plantar regions of the foot and the subungual epithelium present above and around the nail bed (hyponychium, proximal nail fold, matrix and distal groove).

A mixture of solvents, which include benzyl alcohol as a primary carrier, combined with alcohols in an anhydrous phase, or mixed with various amounts of water, and which are still able to retain the highly hydrophobic antifungal agents in solution are used as carriers. This mixture is able to cross both the epidermal barrier working its way through the lipid phase encountered as the solvent migrates through the packed keratinocytes of the epidermis, as well as through the moist nail plate while continuing to carry the drugs in solution. As tissue water begins to dilute the solution the drugs in question precipitate in the interstices of the cells and deposit in a microcrystalline form. It has been shown experimentally that the solutions prepared according the formulas described will hold the antifungal compounds until the water content reaches 40-60%, depending on the compound and the relative concentration of the solvents. Since the water content of the nail ranges between 10 and 30%, the solvent carrier with the terbinafine in solution is able to traverse the nail plate and reach the subungual region, or nail bed. The material, which then begins to precipitate, is deposited in the interstices of the soft tissue and nail plate. It has been shown clinically and experimentally to act as a slow release active principle, thus providing a long-term bioactive function.

The rate of diffusion of water through the nail plate is 10 times greater than through abdominal skin, taken as an example. Unlike what occurs with the stratum corneum of the skin, the permeability coefficient of nails to n-alkanols, an indication of the ability of hydrophobic compounds to traverse a barrier, decreases as the compounds become increasingly hydrophobic. It is therefore apparent that for a compound to traverse the nail plate it has to be water-soluble. It has been postulated that if alkanol permeability could be extrapolated to other low molecular weight organics compounds, very polar compounds might be easily delivered through the nail plate to the underlying tissues (Walters), something that has never been achieved.

Unfortunately all the compounds found to be mycostatic or mycocidal are insoluble or barely soluble in water. Therefore, another carrier must be used.

The solvent system developed and explained in this patent allows the antifungals, in particular terbinafine, an extremely water insoluble molecule, to remain in solution as it traverses through the water environment of the nail plate.

Clinical studies completed using terbinafine alone or combined with ketoconazole and/or griseofulvin, dissolved in the solvent mixtures tested, clearly demonstrated their ability to clear fungal infections in the highly keratinized areas of the plantar region of the foot and under nails affected with onychomycosis, which in the past failed to respond to other forms of delivery.

A total of 10 patients, identified clinically and mycologically (T. rubrum, T. mentagrophytes) were all mycologically cured. Within two weeks of applications all patients with intractable, longstanding (of up to 15 years or more) moccasin distribution fungal dermatomycosis were clinically and mycologically cured. After 2 months of application all patients with severe onychomycosis, also of very long standing duration, appeared mycologically cured (negative cultures). Surprisingly, upon discontinuation of therapy there was no recurrence. Some nails continued to display dystrophic changes commensurate with the slow rate of nail growth and replacement. This persistent and possible prophylactic response is attributed to the unique solubility characteristics of the compounds in question and to the ability of the selected solvents to deliver the drug to the subungual or keratin rich regions where the fungi accumulate, and to persist at such sites in a microcrystalline form after precipitation.

It is believed that the residual protective effects of the terbinafine applied in this solvent mixture, and the lack of any recurrence of what is known to be a very intractable disease, can be attributed to the microcrystalline deposition of the antifungal following its precipitation within the keratinized epidermal tissue as it comes out of solution as the solvent system reaches a critical hydration point.

In a preferred embodiment, the antifungal agent, terbinafine (an allylamine compound) used to treat infections of the nails and of the sub and peri-plantar regions of the foot (moccasin distribution) is dissolved in benzyl alcohol at room temperature with the aid of stirring. The solubility in this compound is very large (greater than 40% w/vol) and this accounts for the properties associated with such a solution. Two grams of terbinafine are therefore dissolved in 5 ml of benzyl alcohol. To this solution 85 ml of ethyl alcohol anhydrous and 10ml of distilled water are added. The final concentration of the active compound terbinafine, is 2% and that of the preferred carrier, benzyl alcohol 5%.

Alternative embodiments include increasing the benzyl alcohol concentration to 10%, using 95% ethanol instead of absolute ethanol, or replacing part of the ethanol with isopropyl alcohol.

Under these conditions the solutions prepared within this framework can be diluted by adding water, while raising the water concentration to up to 45% (v/v) without the active compound coming out of solution. This allows the mixture to traverse the nail plate, where the water content ranges between 10-30% (v/v), without the solute precipitating in its path. Such an ability of the terbinafine to remain in solution until it reaches the nail bed is key to its ability to exhibit its pharmacological activity.

Accordingly, another embodiment of the invention is an antifungal composition comprising an allylamine antifungal compound and an additional antifungal compound. In general, this embodiment of the composition comprises:
(1) an allylamine antifungal compound;
(2) an additional antifungal compound;
(3) an aliphatic alcohol substituted with an aromatic substituent in which the allylamine antifungal compound and the additional antifungal compound are soluble to a degree that a therapeutically effective concentration of the allylamine antifungal compound and of the additional antifungal compound can be applied topically in solution;
(4) a lower aliphatic alcohol in which the aromatic alcohol is soluble; and
(5) water or a water-compatible solvent mixture.

The additional antifungal compound can be any of griseofulvin, ketoconazole, griseofulvin, miconazole, itraconazole, fluconazole, clotrimazole, econazole, terconazole, butoconazole, tioconazole, oxiconazole, sulconazole, cicloprox olamine, haloprogin, and tolnaftate. More than one additional antifungal compound can be used. Typically, the additional antifungal compound is griseofulvin, miconazole, ketoconazole, both griseofulvin and ketoconazole, or both griseofulvin and miconazole. Although terbinafine works effectively on its own, therapeutic efficacy can be improved by combining it with other antifungal compounds that operate by different mechanisms (i.e., cell membrane synthesis inhibitors versus agents that operate by disrupting the cytoskeleton).

In this embodiment, the concentration of the allylamine antifungal compound is preferably about 2% (w/v). The concentration of the additional antifungal compound is typically from about 1% (w/v) to about 3% (w/v), preferably from about 1.5% (w/v) to about 2.5% (w/v), more preferably about 2% (w/v). Other concentrations can be used depending on the specific additional antifungal compounds included. Typically, the volume of the lower aliphatic alcohol used is adjusted to take the concentration of the additional antifungal compound into account. Thus, if one additional antifungal compound is used at a concentration of 2% (w/v), the volume of the lower aliphatic alcohol, such as ethyl alcohol, is reduced by 2%.

Compositions according to the present invention can be applied by conventional methods, including the use of a small brush with a nail-lacquer bottle, a roll-on applicator, or by a squeeze bottle with a small opening. In the results cited above, a small brush with a nail-lacquer bottle was used.

Another embodiment of the invention is a method of treating a fungal infection of skin or nails, particularly onychomycosis. The method comprises topically administering a composition according to the present invention to the skin or nails in an amount therapeutically effective to treat the fungal infection. In particular, the fungal infection is an infection of the plantar or peri-plantar regions of the foot or of the subungual epithelium present above and around the nail bed. The fungal infection can be an infection caused by Trichophyton rubrum, Microsporum canis, T. mentagrophytes, T. interdigitale, or another fungal species. Alternatively, the fungal infection can be tinea pedis (athlete's foot), typically caused by fungi such as T. mentagrophytes, T. rubrum, or Epidermophyton floccosum.

The exact formulation, route of administration and dosage can be chosen by the individual physician in view of the patient's condition. (See, e.g. A. S. Nies & S. P. Spielberg, “Principles of Therapeutics” in J. G. Hardman & L. E. Limbird, eds., “Goodman & Gilman's The Pharmacological Basis of Therapeutics” (9^{th} ed., McGraw-Hill, New York, 1996), ch. 3., pp. 43-62. It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administered dose of a composition according to the present invention will vary with the severity and extent of the fungal infection. Further, the application dose and perhaps the application frequency, can also vary according to the age, body weight, and response of the individual patient, as well as other conditions affecting pharmacodynamic parameters such as liver and kidney function. This should be kept in mind even though systemic absorption of these drugs is relatively low and the total amount of medication used is orders of magnitude less than in the case of systemic administration.

### ADVANTAGES OF THE INVENTION

The present invention provides compositions and methods that are more efficient for treating fungal infections, particularly fungal infections of the skin and nails, by topical application of the compositions without need for systemic administration. Accordingly, the use of compositions and methods according to the present invention minimizes the risk of side effects that can occur with systemic administration of antifungal agents. Some of these side effects, particularly effects on the liver and on the hematopoietic system, can be serious and even life-threatening. At the very least, they can force discontinuance of therapy, leaving the patient without a cure for the fungal infection. The compositions and methods of the present invention avoid this problem. In addition, any recurrence following oral or topical therapy can be easily resolved due to the simplicity and safety of the method described herein.

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference.

### REFERENCES

Dawber R P R, Baran R, structure, embryology, anatomy and physiology of the nail. In Diseases of the Nails. Blackwell Scientific Publications, Oxford, 1984
Nails: Therapy, Diagnosis, Surgery. Scher, R K and Daniel, R C, W B Saunders Company, Philadelphia, 1990
Walters K A. Flynn G L and Marvel J R. Physicochemical characterization of the human nail; Permeation pattern for water and homologous alcohols. J Pharm. Pharmacol. 35: 28,1982

Zaias N, Onychomycosis. Arch. Dermatol. 105: 263 (1972)

Nair B. Final Report on the safety assessment of Benzyl Alcohol Benzoic Acid and Sodium Benzoate. Int J Toxicol. 2001:20 Suppl 3:23-50

U.S. patent Documents:

| | | |
|---|---|---|
| 6,224,887 | May 2001 | Samour et al. |
| 5,889,039 | March 1999 | Knowles |
| 6,585,963 | July 2003 | Quan et al. |
| 6,495,124 | December 2002 | Samour |
| 6,380,236 | April 2002 | Glassman |
| 6,042,845 | March 2000 | Sun et al. |
| 5,487,776 | January 1966 | Nimni |
| 4,820,724 | April 1989 | Nimni |

1. An antifungal composition for topical application to the skin and nails comprising:
(a) an allylamine antifungal compound;
(b) an aliphatic alcohol substituted with an aromatic substituent in which the allylamine antifungal compound is soluble to a degree that a therapeutically effective concentration of the allylamine antifungal compound can be applied topically in solution;
(c) a lower aliphatic alcohol in which the aromatic alcohol is soluble; and
(d) water or a water-compatible solvent mixture.

2. The antifungal composition of claim 1 wherein the allylamine antifungal compound is selected from the group consisting of terbinafine, naftifine, and an analogue or derivative of terbinafine and naftifine.

3. The antifungal composition of claim 2 wherein the allylamine antifungal compound is selected from the group consisting of terbinafine and naftifine.

4. The antifungal composition of claim 3 wherein the allylamine antifungal compound is terbinafine.

5. The antifungal composition of claim 1 wherein the aliphatic alcohol substituted with an aromatic substituent is selected from the group consisting of benzyl alcohol and phenethyl alcohol.

6. The antifungal composition of claim 5 wherein the aliphatic alcohol substituted with an aromatic substituent is benzyl alcohol.

7. The antifungal composition of claim 1 wherein the lower aliphatic alcohol is selected from the group consisting of ethyl alcohol, isopropyl alcohol, and mixtures thereof.

8. The antifungal composition of claim 7 wherein the lower aliphatic alcohol is ethyl alcohol.

9. The antifungal composition of claim 8 wherein the ethyl alcohol is absolute ethyl alcohol.

10. The antifungal composition of claim 8 wherein the ethyl alcohol is 95% ethyl alcohol.

11. The antifungal composition of claim 7 wherein the lower aliphatic alcohol is a mixture of ethyl alcohol and isopropyl alcohol.

12. The antifungal composition of claim 1 wherein the composition comprises water.

13. The antifungal composition of claim 1 wherein the composition comprises a water-compatible solvent mixture.

14. The antifungal composition of claim 1 wherein the concentration of the allylamine antifungal compound is from about 1% (w/v) to about 3% (w/v).

15. The antifungal composition of claim 14 wherein the concentration of the allylamine antifungal compound is from about 1.5% (w/v) to about 2.5% (w/v).

16. The antifungal composition of claim 15 wherein the concentration of the allylamine antifungal compound is about 2% (w/v).

17. The antifungal composition of claim 1 wherein the concentration of the aliphatic alcohol substituted with an aromatic substituent is from about 3% (v/v) to about 10% (v/v).

18. The antifungal composition of claim 17 wherein the concentration of the aliphatic alcohol substituted with an aromatic substituent is about 5% (v/v).

19. The antifungal composition of claim 1 wherein the concentration of the lower aliphatic alcohol is from about 80% (v/v) to about 95% (v/v).

20. The antifungal composition of claim 19 wherein the concentration of the lower aliphatic alcohol is from about 82.5% (v/v) to about 87.5% (v/v).

21. The antifungal composition of claim 20 wherein the concentration of the lower aliphatic alcohol is about 85% (v/v).

22. The antifungal composition of claim 12 wherein the concentration of water is from about 1% (v/v) to about 12.5% (v/v).

23. The antifungal composition of claim 22 wherein the concentration of water is from about 9% (v/v) to about 11% (v/v).

24. The antifungal composition of claim 23 wherein the concentration of water is about 10% (v/v).

25. The antifungal composition of claim 13 wherein the concentration of the water-compatible solvent mixture is from about 1% (v/v) to about 12.5% (v/v).

26. The antifungal composition of claim 25 wherein the concentration of the water-compatible solvent mixture is from about 9% (v/v) to about 11% (v/v).

27. The antifungal composition of claim 26 wherein the concentration of the water-compatible solvent mixture is about 10% (v/v).

28. An antifungal composition for topical application to the skin and nails comprising:
(a) terbinafine in a concentration of about 2% (w/v);
(b) benzyl alcohol in a concentration of about 5% (v/v);
(c) ethyl alcohol in a concentration of about 85% (v/v); and
(d) water in a concentration of about 10% (v/v).

29. An antifungal composition for topical application to the skin and nails comprising:
(a) an allylamine antifungal compound;
(b) an additional antifungal compound;
(c) an aliphatic alcohol substituted with an aromatic substituent in which the allylamine antifungal compound and the additional antifungal compound are soluble to a degree that a therapeutically effective concentration of the allylamine antifungal compound and of the additional antifungal compound can be applied topically in solution;
(d) a lower aliphatic alcohol in which the aromatic alcohol is soluble; and
(e) water or a water-compatible solvent mixture.

30. The antifungal composition of claim 29 wherein the additional antifungal compound is selected from the group consisting of griseofulvin, ketoconazole, griseofulvin, miconazole, itraconazole, fluconazole, clotrimazole, econazole, terconazole, butoconazole, tioconazole, oxiconazole, sulconazole, cicloprox olamine, haloprogin, and tolnaftate.

31. The antifungal composition of claim 30 wherein the additional antifungal compound is griseofulvin.

32. The antifungal composition of claim 30 wherein the additional antifungal compound is miconazole.

33. The antifungal composition of claim 30 wherein the additional antifungal compound is ketoconazole.

34. The antifungal composition of claim 30 wherein the additional antifungal compound is griseofulvin and ketoconazole.

35. The antifungal composition of claim 30 wherein the additional antifungal compound is griseofulvin and miconazole.

36. The antifungal composition of claim 29 wherein the allylamine antifungal compound is selected from the group consisting of terbinafine, naftifine, and an analogue or derivative of terbinafine and naftifine.

37. The antifungal composition of claim 36 wherein the allylamine antifungal compound is selected from the group consisting of terbinafine and naftifine.

38. The antifungal composition of claim 37 wherein the allylamine antifungal compound is terbinafine.

39. The antifungal composition of claim 29 wherein the aliphatic alcohol substituted with an aromatic substituent is selected from the group consisting of benzyl alcohol and phenethyl alcohol.

40. The antifungal composition of claim 39 wherein the aliphatic alcohol substituted with an aromatic substituent is benzyl alcohol.

41. The antifungal composition of claim 29 wherein the lower aliphatic alcohol is selected from the group consisting of ethyl alcohol, isopropyl alcohol, and mixtures thereof.

42. The antifungal composition of claim 41 wherein the lower aliphatic alcohol is ethyl alcohol.

43. The antifungal composition of claim 42 wherein the ethyl alcohol is absolute ethyl alcohol.

44. The antifungal composition of claim 42 wherein the ethyl alcohol is 95% ethyl alcohol.

45. The antifungal composition of claim 41 wherein the lower aliphatic alcohol is a mixture of ethyl alcohol and isopropyl alcohol.

46. The antifungal composition of claim 29 wherein the concentration of the allylamine antifungal compound is from about 1% (w/v) to about 3% (w/v).

47. The antifungal composition of claim 46 wherein the concentration of the allylamine antifungal compound is from about 1.5% (w/v) to about 2.5% (w/v).

48. The antifungal composition of claim 47 wherein the concentration of the allylamine antifungal compound is about 2% (w/v).

49. The antifungal composition of claim 29 wherein the concentration of the additional antifungal compound is from about 1% (w/v) to about 3% (w/v).

50. The antifungal composition of claim 49 wherein the concentration of the additional antifungal compound is about 2% (w/v).

51. The antifungal composition of claim 29 wherein the concentration of the aliphatic alcohol substituted with an aromatic substituent is from about 3% (v/v) to about 10% (v/v).

52. The antifungal composition of claim 51 wherein the concentration of the aliphatic alcohol substituted with an aromatic substituent is about 5% (v/v).

53. The antifungal composition of claim 29 wherein the concentration of the lower aliphatic alcohol is from about 80% (v/v) to about 95% (v/v).

54. The antifungal composition of claim 29 wherein the composition comprises water.

55. The antifungal composition of claim 29 wherein the composition comprises a water-compatible solvent mixture.

56. The antifungal composition of claim 54 wherein the concentration of water is from about 1% (v/v) to about 12.5% (v/v).

57. The antifungal composition of claim 56 wherein the concentration of water is from about 9% (v/v) to about 11% (v/v).

58. The antifungal composition of claim 57 wherein the concentration of water is about 10% (v/v).

59. The antifungal composition of claim 55 wherein the concentration of the water-compatible solvent mixture is from about 1% (v/v) to about 12.5% (v/v).

60. The antifungal composition of claim 59 wherein the concentration of the water-compatible solvent mixture is from about 9% (v/v) to about 11% (v/v).

61. The antifungal composition of claim 60 wherein the concentration of the water-compatible solvent mixture is about 10% (v/v).

62. A method for treatment of a fungal infection of skin or nails comprising administering the antifungal composition of claim 1 topically to the skin or nails in an amount therapeutically effective to treat the fungal infection.

63. The method of claim 62 wherein the fungal infection is an infection of the plantar or peri-plantar regions of the foot.

64. The method of claim 62 wherein the fungal infection is an infection of the subungual epithelium present above and around the nail bed.

65. The method of claim 62 wherein the fungal infection is caused by a fungus selected from the group consisting of Trichophyton rubrum, Microsporum canis, T. mentagrophytes, T. interdigitale, T. rubrum, and Epidermophyton floccosum.

66. The method of claim 62 wherein the fungal infection is tinea pedis.

67. The method of claim 62 wherein the allylamine antifungal compound is terbinafine.

68. The method of claim 62 wherein the aliphatic alcohol substituted with an aromatic substituent is benzyl alcohol.

69. The method of claim 62 wherein the lower aliphatic alcohol is ethyl alcohol.

70. A method for treatment of a fungal infection of skin or nails comprising administering the antifungal composition of claim 28 topically to the skin or nails in an amount therapeutically effective to treat the fungal infection.

71. The method of claim 70 wherein the fungal infection is an infection of the plantar or peri-plantar regions of the foot.

72. The method of claim 70 wherein the fungal infection is an infection of the subungual epithelium present above and around the nail bed.

73. The method of claim 70 wherein the fungal infection is caused by a fungus selected from the group consisting of Trichophyton rubrum, Microsporum canis, T. mentagrophytes, T. interdigitale, T. rubrum, and Epidermophyton floccosum.

74. The method of claim 70 wherein the fungal infection is tinea pedis.

75. A method for treatment of a fungal infection of skin or nails comprising administering the antifungal composition of claim 29 topically to the skin or nails in an amount therapeutically effective to treat the fungal infection.

76. The method of claim 75 wherein the fungal infection is an infection of the plantar or peri-plantar regions of the foot.

77. The method of claim 75 wherein the fungal infection is an infection of the subungual epithelium present above and around the nail bed.

78. The method of claim 75 wherein the fungal infection is caused by a fungus selected from the group consisting of Trichophyton rubrum, Microsporum canis, T. mentagrophytes, T. interdigitale, T. rubrum, and Epidermophyton floccosum.

79. The method of claim 75 wherein the fungal infection is tinea pedis.

80. The method of claim 75 wherein the allylamine antifungal compound is terbinafine.

81. The method of claim 75 wherein the aliphatic alcohol substituted with an aromatic substituent is benzyl alcohol.

82. The method of claim 75 wherein the lower aliphatic alcohol is ethyl alcohol.

83. The method of claim 75 wherein the additional antifungal compound is griseofulvin.

84. The method of claim 75 wherein the additional antifungal compound is miconazole.

85. The method of claim 75 wherein the additional antifungal compound is ketoconazole.

86. The method of claim 75 wherein the additional antifungal compound is griseofulvin and ketoconazole.

87. The method of claim 75 wherein the additional antifungal compound is griseofulvin and miconazole.
